(19) United States
(12) Patent Application Publication
(10) Pub. No.: US 2026/0357220 A1
(43) Pub. Date: Nov. 12, 2026

(54) THREE-DIMENSIONAL SKIN MODEL

(71) Applicant: ANDS CORPORATION, Osaka-shi, Osaka (JP)

(72) Inventors: Katsunari TEZUKA, Osaka-shi, Osaka (JP); Hiroaki SHICHIRI, Osaka-shi, Osaka (JP)

(21) Appl. No.: 19/124,930

(22) PCT Filed: Oct. 26, 2023
(86) PCT No.: PCT/JP2023/038677

§ 371 Date: Apr. 28, 2025

(30) Foreign Application Priority Data
Oct. 27, 2022 (JP) 2022-172729

(51) Int. Cl. C12N 5/071; C12M 1/32; C12M 3/00; C12N 5/077; G01N 33/50

(52) U.S. Cl. CPC C12N 5/0698; C12M 21/08; C12M 23/12; C12N 5/0629; C12N 5/0656; G01N 33/5082; C12N 2513/00; C12N 2533/54

(57) ABSTRACT

A three-dimensional skin model according to one embodiment of the present invention comprises: a first layer including keratinocytes or cells with differentiated keratinocytes; a second layer including dermal fibroblasts; and an intermediate membrane which is interposed between the first layer and the second layer. The intermediate membrane is a porous membrane including, at least on the surface thereof, an extracellular matrix. Further, a three-dimensional skin model according to another embodiment of the present invention comprises an epidermal layer, a dermal layer, and an intermediate membrane which is interposed between the epidermal layer and the dermal layer. The epidermal layer has a stratum corneum, a stratum granulosum, a stratum spinosum, and a stratum basale. The dermal layer has a multi-layer structure and has a cellular density of no less than 2,000 cells/cm².

### TECHNICAL FIELD

The present invention relates to a three-dimensional skin model.

### BACKGROUND ART

With the growing momentum of animal care worldwide, animal experimental alternatives are being developed. As an alternative to animal experiments of skin, artificial skin models such as a chemical fiber skin, a three-dimensional skin model, etc. have been developed instead of human skin, which is difficult to obtain.

For example, in a functional test of products (cosmetic ingredients and the like), a full-thickness skin model capable of checking responsiveness of the epidermis and dermis has been developed. The full-thickness skin model is a skin model in which a collagen hydrogel is used as an extracellular matrix; epidermal keratinocytes are seeded on a dermal layer, in which dermal fibroblasts in the collagen hydrogel are dispersed; and the epidermal layer is reconstructed by vapor phase culture.

Here, since the dermal fibroblasts shrink the collagen hydrogel, the epidermal layer is peeled off from the cell culture insert during culture. Thus, an evaluation method of directly applying a test substance (cosmetic component or the like) from the stratum corneum side is difficult. Further, since the shrinkage of the dermal layer occurs during culture of the skin model, if a culture period becomes long, the morphology of the skin model and the responsiveness of the skin may be affected. It is, therefore, necessary to study a skin model capable of controlling shrinkage of a dermal layer.

Thus, there is disclosed a skin model in which a polyethylene terephthalate film is disposed between the epidermal layer and the dermal layer such that the influence of shrinkage of the dermal layer on the epidermal layer is alleviated (Non-Patent Document 1).

### CITATION LIST

### Non-Patent Document

Non-Patent Document 1: Freia F. Schmidt, Sophia Nowakowski and Petra J. Kluger, Frontiers in Bioengineering and Biotechnology, 8 May 2020, Volume 8, Article 388: 1-11.

### DISCLOSURE OF THE INVENTION

### Problems to be Solved by the Invention

However, since the skin model disclosed in Non-Patent Document 1 uses a collagen hydrogel as a matrix in the dermal layer, it is difficult to sufficiently suppress shrinkage of the dermal layer.

In view of the foregoing, an object of the present invention is to provide a three-dimensional skin model capable of suppressing shrinkage of the dermal layer and maintaining the structure of the epidermis and dermis for a long period of time.

### Means for Solving the Problems

As a result of diligent studies, the present inventors have found that the above problems can be solved, and have completed the present invention. Specifically, the present invention is configured as in the following (1) to (12).
(1) A three-dimensional skin model including: a first layer including keratinocytes or cells differentiated from keratinocytes; a second layer including dermal fibroblasts; and an intervening membrane interposed between the first layer and the second layer, in which the intervening membrane is a porous membrane including at least an extracellular matrix on a surface thereof.
(2) The three-dimensional skin model as described in (1), in which the three-dimensional skin model comprises a collagen hydrogel in an amount of 0 w/v % or more and less than 0.6 w/v %, and v refers to a volume of the second layer.
(3) A three-dimensional skin model, including: an epidermal layer; a dermal layer; and an intervening membrane interposed between the epidermal layer and the dermal layer, in which the epidermal layer has a stratum corneum, a stratum granulosum, a stratum spinosum, and a stratum basale, and the dermal layer has a multilayer structure and has a cell density of 2,000 cells/cm² or more.
(4) A three-dimensional skin model, including: an epidermal layer; a dermal layer; and an intervening membrane interposed between the epidermal layer and the dermal layer, in which the epidermal layer has a stratum corneum, a stratum granulosum, a stratum spinosum, and a stratum basale, the dermal layer has a multilayer structure and includes a collagen hydrogel in an amount of 0 w/v % or more and less than 0.6 w/v %, and v refers to a volume of the dermal layer.
(5) A culture container including: the three-dimensional skin model as described in any one of (1) to (4); a plurality of wells containing a culture fluid; and a support portion that supports the three-dimensional skin model in each well and allows the culture fluid to communicate with the three-dimensional skin model on a second layer side or dermal layer side thereof.
(6) The culture container as described in (5), in which the culture container has 12, 24, 48, or 96 wells.
(7) A method of evaluating a sample, including: a step of culturing a three-dimensional skin model using the culture container as described in (5) or (6); a step of administering a sample to the three-dimensional skin model during culture; and a step of evaluating a function given to the three-dimensional skin model by the sample by analyzing the three-dimensional skin model or a culture supernatant after a predetermined culture period elapses from the administration of the sample.
(8) The method for evaluating as described in (7), in which the culture period is 6 days or more.
(9) The method for evaluating as described in (7) or (8), in which the step of evaluating includes, during the culture period, a step of processing the three-dimensional skin model, a step of administering same samples or different samples two or more times, or a step of removing the administered samples.
(10) The method for evaluating as described in any one of (7) to (9), in which the three-dimensional skin model to which at least a sample is not administered has an epidermal layer having a stratum corneum, a stratum granulosum, a stratum spinosum, and a stratum basale, and has a dermal layer having a multilayer structure, after the culture period elapses.
(11) The method for evaluating as described in any one of (7) to (10), in which a plane area of an epidermal layer and a plane area of a dermal layer of the three-dimensional skin model after the culture period elapses satisfy a relationship of the plane area of the epidermal layer≤the plane area of the dermal layer, or the plane area of the dermal layer after the culture period elapses is 0.7 times or more the plane area of the dermal layer before the culture period elapses.
(12) The method for evaluating as described in any one of (7) to (11), in which the analysis includes gene expression, protein expression, histochemical analysis, or skin barrier function analysis.

### Effects of the Invention

According to the present invention, it is possible to provide a three-dimensional skin model capable of suppressing shrinkage of the dermal layer and maintaining the structure of the epidermis and dermis over a long period of time.

### BRIEF DESCRIPTION OF THE DRAWINGS

FIG. 1 is a skin tissue slice image of a three-dimensional skin model after hematoxylin-eosin staining;

FIG. 2A is a plan view of a culture container having a plurality of wells, and FIG. 2B is a cross-sectional view taken along line A-A of (a);

FIGS. 3A and 3B are skin tissue slice images of a three-dimensional skin model after culture for a predetermined period of time;

FIGS. 4A and 4B are skin tissue slice images of a three-dimensional skin model subjected to immunohistochemistry staining;

FIG. 5 is a graph showing an analysis result of a barrier function of a three-dimensional skin model;

FIG. 6 is a graph showing an analysis result of a barrier function of a three-dimensional skin model;

FIGS. 7A and 7B are graphs showing analysis results of responsiveness of a three-dimensional skin model to a physiological stimulus;

FIGS. 8A and 8B are graphs showing analysis results of responsiveness to a physiological stimulus of a three-dimensional skin model;

FIGS. 9A and 9B are skin tissue slice images after analysis of the responsiveness of a three-dimensional skin model to a physiological stimulus;

FIGS. 10A to 10D are graphs showing analysis results of responsiveness of a three-dimensional skin model to cosmetics;

FIGS. 11A to 11D are graphs showing analysis results of responsiveness of a three-dimensional skin model to cosmetics;

FIGS. 12A and 12B are graphs showing analysis results of protein expression;

FIGS. 13A to 13F are graphs showing analysis results of responsiveness to commercially available cosmetics (serum);

FIGS. 14A to 14F are graphs showing analysis results of responsiveness to commercially available cosmetics (lotions);

FIGS. 15A to 15F are graphs showing analysis results of responsiveness to commercially available cosmetics (emulsions); and

FIGS. 16A to 16F are graphs showing analysis results of responsiveness to commercially available cosmetics (creams).

### PREFERRED MODE FOR CARRYING OUT THE INVENTION

Hereinafter, specific embodiments of the present invention will be described in detail. It should be noted that the present invention is not limited to the following embodiments, and can be appropriately modified without departing from the gist of the present invention.

### Three-Dimensional Skin Model

### (First Embodiment)

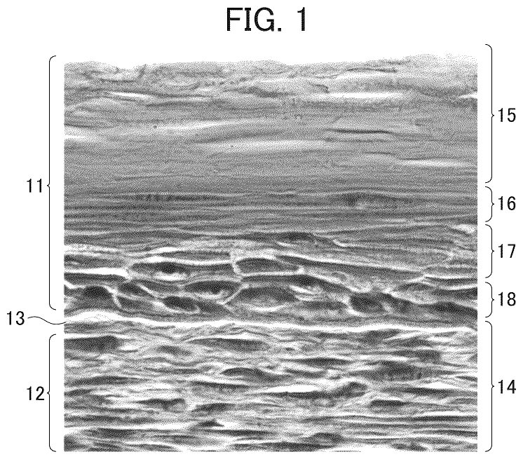

As shown in FIG. 1, a three-dimensional skin model according to a first embodiment of the present invention includes a first layer 11 containing keratinocytes or cells differentiated from keratinocytes, a second layer 12 containing dermal fibroblasts, and an intervening membrane 13 interposed between the first layer 11 and the second layer 12. The intervening membrane 13 includes an extracellular matrix. The second layer 12 preferably contains a collagen hydrogel in an amount of 0 w/v % or more and less than 0.6 w/v %. Here, v refers to a volume of the second layer.

Hereinafter, each constituent element will be described.

### <<First Layer>>

The first layer 11 includes keratinocytes or cells differentiated from keratinocytes. Examples of the cells differentiated from keratinocytes include one or more selected from the group consisting of corneocytes, granular cells, spinous cells, and basal cells described below.

Keratinocytes are epidermal cells that divide in the stratum basale, which is the lowermost layer of the epidermis, into the skin surface.

### <<Second Layer>>

The second layer 12 includes dermal fibroblasts.

Dermal fibroblasts (fibroblasts) 14 are cells that produce collagen fibers, elastic fibers, mucopolysaccharides, and the like, and exhibit an elongated spindle-shaped form.

The second layer 12 preferably contains a collagen hydrogel in an amount of 0 w/v % or more and less than 0.6 w/v %, and more preferably in an amount of 0 w/v % or more and 0.3 w/v % or less. When the amount of the collagen hydrogel is within the above range, shrinkage of the dermal layer constituting the three-dimensional skin model during culture can be suppressed.

In addition, the proliferative potential of dermal fibroblasts by inhibition of cell activity is less likely to be inhibited, and the proliferation of dermal fibroblasts is less likely to be inhibited because space in which the proliferated cells can exist is spacious. Therefore, the cell density of a dermal layer is likely to be maintained at a high level over a long period of time, which makes it easier to evaluate the effect of a sample on the dermal layer with high sensitivity. Further, as a result of proteins (cytokines, chemokines, and the like), lipid components (eicosanoids, and the like), and the like involved in crosstalk being produced and transmitted to the epidermal layer, a differentiated structure of the epidermal layer that constitutes the three-dimensional skin model in culture is likely to be maintained to a high degree over a long period of time.

### <<Intervening Membrane>>

The intervening membrane 13 is a porous membrane that allows a substance to permeate therethrough, and includes at least an extracellular matrix on a surface thereof. The inclusion of an intervening membrane on at least the surface of the extracellular matrix facilitates keratinocytes and/or fibroblasts to adhere without reliance on collagen hydrogels.

The intervening membrane may consist essentially of an extracellular matrix layer alone or may include an extracellular matrix on the surface of the substrate. The extracellular matrix on the surface is not particularly limited, but is preferably a coating film because of its high frequency of existence.

Examples of the material of the base material include a film of PET (polyethylene terephthalate) and/or PC (polycarbonate), and PET. Examples of the extracellular matrices include type I collagen, type III collagen, type IV collagen, fibronectin, gelatin, and the like.

An average pore diameter of the intervening membrane is preferably 0.4 μm or more. The average pore diameter of the intervening membrane may be 8.0 μm or less, and is preferably 6.0 μm or less. When the average pore diameter of the intervening membrane is within the above range, the permeation of a protein (cytokines, chemokines, etc.) and a lipid component (eicosanoids, etc.) involved in crosstalk can proceed well. The pore diameter of the intervening membrane is obtained, for example, by measuring a major axis using an image of an electron microscope.

An average thickness of the intervening membrane is preferably 20 μm or less, more preferably 15 μm or less, 10 μm or less, 7.5 μm or less, or 5 μm or less, from the viewpoint that a skin model in which the structure of the epidermis and dermis are maintained over a long period of time can be easily obtained without reliance on medium components (substances involved in crosstalk such as KGF, HGF, IGF-1, etc.) or an additional layer described below. However, when a specific medium component (substances involved in crosstalk such as KGF, HGF, IGF-1, etc.) is used and/or an additional layer is provided, an average thickness of the intervening membrane may be, for example, 20 μm or more and 200 μm or less. When the thickness of the intervening membrane is within the above range, strength of the three-dimensional skin model is increased, and the crosstalk between the dermal layer and the epidermal layer is performed to a high degree, so that a state in which the differentiation of the epidermal layer proceeds appropriately (the stratum spinosum and the stratum granulosum become thick) and/or a state in which density of the stratum basale is high are/is easily maintained. The thickness of the intervening membrane can be measured, for example, using optical microscopy images of histochemical sections.

As the intervening membrane, a membrane of a commercially available cell culture insert may be used.

The three-dimensional skin model according to the present embodiment may or may not include another layer in addition to the first layer, the second layer, and the intervening membrane. Examples of the additional layer include a subcutaneous tissue layer, such as a fat layer located opposite the intervening membrane of the second layer.

When evaluating the additional layer itself, it is preferable to have the additional layer. On the other hand, an embodiment having the additional layer further promotes crosstalk and facilitates maintaining the differentiated structure, while a degree of freedom of culture conditions (e.g., medium composition) suitable for all of the first layer, the second layer, and the additional layer is low. In the present embodiment, since the differentiated structure can be easily maintained without reliance on an additional layer, an embodiment without the additional layer is preferable.

### (Second Embodiment)

As illustrated in FIG. 1, the three-dimensional skin model according to the second embodiment of the present invention includes an epidermal layer 11, a dermal layer 12, and an intervening membrane 13 interposed between the epidermal layer 11 and the dermal layer 12. The epidermal layer 11 includes a stratum corneum 15, a stratum granulosum 16, a stratum spinosum 17, and a stratum basale 18.

Hereinafter, each constituent element will be described.

### <<Epidermal Layer>>

The epidermal layer 11 has a four-layer structure in which stratum corneum cells form the stratum corneum 15 in the uppermost layer, granular cells form the stratum granulosum 16 just below the stratum corneum 15, spinous cells (or thorn cells) form the stratum spinosum 17 just below the stratum granulosum 16, and basal cells differentiated from keratinocytes form the stratum basale 18 just below the stratum spinosum 17 (lowermost layer). The epidermal layer 11 may include pigment cells (melanocytes).

### <Stratum Corneum>

As described above, the stratum corneum 15 is located in the uppermost layer of the epidermal layer 11 that constitutes the three-dimensional skin model, and is configured by a layered structure constructed of corneocytes. In addition, the corneocytes have a flat shape, and their nuclei have disappeared during differentiation. In addition, corneocytes have keratin fibrils aggregated in their cytoplasm, are acidophilic, and are stained light red to dark red by hematoxylin-eosin (HE) staining.

### <Stratum Granulosum>

As described above, the stratum granulosum 16 is located directly below the stratum corneum 15 and directly above the stratum spinosum 17, to be described later, and has a layered structure constructed of granular cells. The granular cells have a flat shape and contain granules composed of a basophilic component in the cells. Intracellular granules are stained blue-violet to light blue by hematoxylin-eosin (HE) staining. The stratum granulosum of the three-dimensional skin model according to the present embodiment preferably has a thickness of 3 μm or more on average, more preferably 6 μm or more on average, and still more preferably 8 μm or more on average. The thickness of the stratum granulosum of the three-dimensional skin model is preferably 3 μm or more and 10 μm or less on average. The thickness can be measured, for example, using optical microscopy images of histochemical sections.

### <Stratum Spinosum>

As described above, the stratum spinosum 17 is located directly below the stratum granulosum 16 and directly above the stratum basale 18, which will be described later, and is constituted by a layered structure constructed of spinous cells. The spinous cells have a flat shape, and have a structure in which spines are arranged around the cells. The spines can be confirmed by hematoxylin-eosin (HE) staining. The stratum spinosum of the three-dimensional skin model according to the present embodiment has a thickness of preferably 18 μm or more on average, more preferably 26 μm or more on average, and still more preferably 39 μm or more on average. The thickness of the stratum spinosum of the three-dimensional skin model is preferably 18 μm or more and 50 μm or less on average. The thickness can be measured, for example, using optical microscopy images of histochemical sections.

### <Stratum Basale>

As described above, the stratum basale 18 is located in the lowermost layer of the epidermal layer 11, and is constituted by a layered structure constructed of basal cells. Unlike cornified cells, granular cells, and spinous cells, the basal cells have a cubic to columnar shape and have an elliptical nucleus. Basal cells are basophilic and stained indigo to light blue by hematoxylin-eosin (HE) staining. The stratum basale of the three-dimensional skin model according to the present embodiment preferably has a thickness of 7 μm or more on average, more preferably 9 μm or more on average, and still more preferably 14 μm or more on average. The thickness of the stratum spinosum of the three-dimensional skin model is preferably 7 μm or more and 20 μm or less on average. Thickness can be measured, for example, using optical microscopy images of histochemical sections.

### Dermal Layer

The dermal layer 12 has a multilayer structure. Specifically, the dermal layer 12 has a tissue structure composed of fibroblasts, collagen fibers (such as collagen) synthesized and secreted by fibroblasts, elastic fibers (such as fibrillin and elastin), and other extracellular matrix components (such as hyaluronic acid).

The “multilayer structure” refers to a structure in which fibroblasts form two or more layers in a vertical direction (stratum corneum direction). Here, the orientation of the fibroblasts is not constant. Therefore, the fibroblasts are oriented in the lateral direction in some cases, whereby the skin tissue section image sliced in the vertical direction has a spindle shape, or the fibroblasts are oriented in the longitudinal direction in some cases, whereby the skin tissue section image has a circular shape.

The dermal layer (dermal fibroblasts) has a cell density of 2,000 cells/cm² or more, preferably 5,000 cells/cm² or more, and most preferably 6,000 cells/cm² or more. The cell density of the dermal layer is preferably 2,000 cells/cm² or more and 10,000 cells/cm² or less, more preferably 5,000 cells/cm² or more and 8,000 cells/cm² or less, and further preferably 6,000 cells/cm² or more and 7,700 cells/cm² or less. When the cell density of the dermal layer is within the above range, the layered structure of the three-dimensional skin model can be maintained for a long period of time.

### (Third Embodiment)

As illustrated in FIG. 1, the three-dimensional skin model according to the third embodiment of the present invention includes the epidermal layer 11, the dermal layer 12, and the intervening membrane 13 interposed between the epidermal layer 11 and the dermal layer 12. The epidermal layer 11 includes the stratum corneum 15, the stratum granulosum 16, the stratum spinosum 17, and the stratum basale 18.

The dermal layer 12 has a multilayer structure and contains a collagen hydrogel in an amount of 0 w/v % or more and less than 0.6 w/v %. Here, v refers to a volume of the dermal layer. More preferably, the dermal layer 12 contains a collagen hydrogel in an amount of 0 w/v % or more and 0.3 w/v % or less. Alternatively, the dermal layer 12 is not in a state in which collagen fibers are unevenly dispersed by hematoxylin-eosin (HE) staining. As a result, proliferative potential of dermal fibroblasts is less likely to be inhibited because the space in which proliferated cells can exist is spacious. Therefore, shrinkage of the dermal layer constituting the three-dimensional skin model during culture can be suppressed.

In addition, the proliferative potential of dermal fibroblasts is less likely inhibited by cell activity inhibition, and the space in which proliferated cells can exist is spacious, whereby the proliferation of dermal fibroblasts is less likely to be inhibited. Therefore, the cell density of the dermal layer is likely to be maintained at a high level over a long period of time, which makes it easier to evaluate the effect of the sample on the dermal layer with high sensitivity. As a result of proteins (cytokines, chemokines, and the like), lipid components (eicosanoids, and the like), and the like involved in crosstalk being produced and transmitted to the epidermal layer, the differentiated structure of the epidermal layer constituting the three-dimensional skin model during culture is likely to be maintained to a high degree over a long period of time.

The three-dimensional skin model according to the present embodiment may or may not have another layer in addition to the epidermal layer, the dermal layer, and the intervening membrane. Additional layers include a subcutaneous tissue layer, such as a fat layer located opposite the intervening membrane of the dermal layer.

An embodiment including an additional layer further promotes crosstalk and facilitates maintaining the differentiated structure, while reducing the degree of freedom in culture conditions (e.g., media composition), which are suitable for all of the epidermal, dermal, and the additional layer. In the present embodiment, since maintaining the differentiated structure is easy without reliance on the additional layer, an embodiment without an additional layer is preferable.

### Culture Container

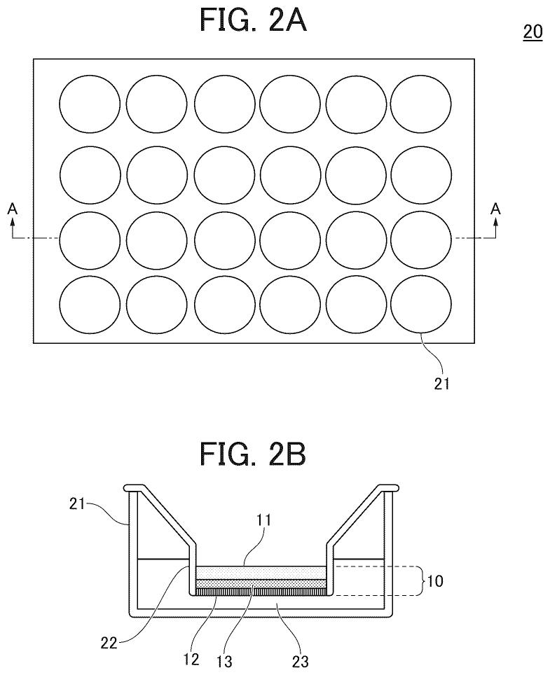

As illustrated in FIGS. 2A and 2B, a culture container 20 used in the first to third embodiments includes a plurality of wells 21 that accommodate a culture fluid 23, and a support portion 22 that supports a three-dimensional skin model 10 in each of the wells 21. The three-dimensional skin model 10 is disposed so as to be able to communicate with the culture fluid 23 on a second layer 12 side or a dermal layer 12 side.

The culture container 20 may have any number of wells, including but not limited to 12, 24, 48, or 96 wells. As a result, evaluations regarding a large number of samples and application conditions can be performed at the same time.

### Sample Evaluation Method

The sample evaluation method of the present invention is performed using the culture container. Specifically, the sample evaluation method includes (1) a step of culturing a three-dimensional skin model using the culture container 20 shown in FIGS. 2A and 2B (culture step); (2) a step of administering a sample to the three-dimensional skin model during culture (sample administration step); and (3) a step of evaluating the function given to the three-dimensional skin model by the sample by analyzing the three-dimensional skin model or a culture supernatant after a predetermined culture period elapses from the administration of the sample (evaluation step).

Hereinafter, each step will be described.

### (Culture Step)

More specifically, the culture step is a step of disposing the three-dimensional skin model 10 in the well 21 of the culture container 20, so as to be able to communicate with the culture fluid 23 on a second layer 12 side or a dermal layer 12 side of the three-dimensional skin model 10, and culturing the three-dimensional skin model 10 for a predetermined period of time.

The culture period is 6 days or more, 12 days or more, and more preferably 20 days or more. The culture period may be 30 days or less. When the culture period is 6 days or more, the period during which evaluation is possible is long, so that a sufficient evaluation result can be obtained.

### (Sample Administration Step)

More specifically, the sample administration step is a step of administering a sample to the three-dimensional skin model 10 disposed in the well 21 of the culture container 20 by a predetermined method.

The sample to be administered is not particularly limited, but may be an isolated one or two or more substances, or may be a mixture of various ingredients (e.g., cosmetics, pharmaceuticals). The form of the sample is not particularly limited, and may be a liquid, a gel, a cream, a powder, or the like. The method of administering the sample is not particularly limited, and the sample may be applied to the surface of the epidermis, may be injected into the epidermis, may be injected into the dermis, or may be added to the culture fluid.

In addition, samples which are different from each other may be applied to each well, or the same sample may be applied by different methods (dose and/or usage). This makes it possible to quantitatively compare the performance of a sample on the skin and/or to infer a preferred mode of administration (dose and/or usage) to provide the skin with a desired function.

### (Evaluation Step)

More specifically, the evaluation step is a step of evaluating the function of a sample on the three-dimensional skin model 10 by analyzing the three-dimensional skin model 10 or the culture supernatant containing a secretion fluid or the like secreted by the cells after a culture period of several hours or more elapses from the administration of the sample.

The analysis preferably includes gene expression, protein expression, histochemical analysis, or skin barrier function analysis, and may include a combination of two or more thereof.

The evaluation step may include a step of processing the three-dimensional skin model 10, a step of administering the same or different samples to the three-dimensional skin model 10 two or more times, or a step of removing the samples administered to the three-dimensional skin model 10, each step being performed in the above-described culture period.

The step of processing the three-dimensional skin model 10 is a step of preparing three-dimensional skin models in different states. The method of processing is not particularly limited, and includes addition of an inflammatory substance for inducing oxidative stress, irradiation with a light beam for inducing oxidative stress such as UV, and the like. Although this processing may take several seconds to several days, since the skin model used in the present embodiment can maintain the structure of the epidermal layer and the dermal layer for a long time, evaluation using the processed skin model can be freely performed. By preparing three-dimensional skin models in different states, not only the evaluation of the skin in a normal state but also the evaluation of the skin in a rough skin state, inflamed skin, and the like can be performed, and thus, the efficacy, etc. of the sample to various skin states can be acquired.

In the step of administering the same or different samples two or more times, the same sample may be administered in the same mode or in different mode. Also, different samples may be administered in the same mode or in different modes. As used herein, the term “mode” refers to a dose and/or usage.

In addition, for comparison with the three-dimensional skin model to which a sample has been administered, a three-dimensional skin model to which at least the sample is not administered is subjected to the above-described culture step for a predetermined period of time (for example, the culture period may be 6 days or more, and preferably 12 days or more, or 20 days or more, and 30 days or less). Here, the culture is preferably performed under a condition that the three-dimensional skin model to which a sample is not administered has an epidermal layer having a stratum corneum, a stratum granulosum, a stratum spinosum, and a stratum basale, and a dermal layer having a multilayer structure after the culture period elapses.

After the culture period elapses, a plane area of the epidermal layer and a plane area of the dermal layer of the three-dimensional skin model preferably satisfy the following relationship: the plane area of the epidermal layer≤the plane area of the dermal layer. Alternatively, the plane area of the dermal layer after the culture period elapses is preferably 0.7 times or more, and more preferably 0.9 times or more, though not particularly being limited, with respect to the plane area of the dermal layer before the culture period elapses. Moreover, the plane area of the dermal layer after the culture period elapses is preferably 1.0 times or less the plane area of the dermal layer before the culture period elapses. When the relationship between the plane area of the epidermal layer and the plane area of the dermal layer of the three-dimensional skin model is within the above range, shrinkage of the dermal layer during culture is suppressed, and thus evaluation over a long period of time becomes possible. The plane area of the epidermal layer is preferably 0.7 times or more and 1.0 times or less, more preferably 0.9 times or more and 1.0 times or less, with respect to the plane area of the dermal layer before the culture period elapses.

### EXAMPLES

Hereinafter, the present invention will be described in more detail with reference to Examples, but the present invention is not limited to the following Examples.

### Construction of Three-Dimensional Skin Model

A cell culture insert (ad-MED Vitrigel 2.08364-96 (manufactured by Kanto Chemical Co., Inc.) and AteloCell CM-24 (manufactured by Koken Co., Ltd.)) having a collagen membrane was prepared. On one side of the cell culture insert, 10,000 normal human dermal fibroblasts suspended in a 10% FBS-containing DMEM were seeded and cultured in a CO₂ incubator at 37 °C for 1 day. Next, 500,000 normal human epidermal keratinocytes suspended in Humedia KG2 (manufactured by Kurabo) were seeded on the other surface of the cell culture insert, cultured in the CO₂ incubator at 37 °C for 1 day, the medium was replaced by a medium for three-dimensional culture, and culture was further continued for 1 day. After culture, the medium on the normal human keratinized epidermal cell side was removed, and vapor phase culture was started. Epidermal layer differentiation was induced by performing the vapor phase culture for 12 days, and a three-dimensional skin model was constructed. It should be noted that “Vitrigel” is a registered trademark of National Agriculture and Food Research Organization, “AteloCell” is that of Koken Co., Ltd., and “Humedia” is that of Kurabo Industries Ltd.

The constructed three-dimensional skin model had thicknesses of the stratum granulosum, stratum spinosum, and stratum basale of 3 to 8 μm, 18 to 39 μm, and 7 to 14 μm, respectively. The dermal layer had a cell density of 7114±339 cells/cm², and had a cell density of 5843±349 cells/cm² after vapor phase culture was carried out for 20 days. According to the search by the present inventors, the cell density of the dermal layer of the model disclosed in Non-Patent Document 1 was 1300±339 cells/cm², which was significantly lower than that of the present Example. The plane areas of the dermal layer and the epidermal layer of the constructed three-dimensional skin model after the culture period elapsed were 1 time the plane areas of the dermal layer and the epidermal layer before the culture period elapsed, respectively.

### Histochemical Analysis 1 of Three-Dimensional Skin Model

### (Analysis Method)

A skin tissue of the three-dimensional skin model, which had been cultured in the vapor phase for 12 days by the above-described method, was fixed with Superfix KY-500 (manufactured by Kurabo Industries Ltd.) and embedded in Paraplast X-tra (manufactured by Leica Biosystems) to prepare a paraffin-embedded section (skin tissue section). The skin tissue section was stained with hematoxylin-eosin (HE) staining, and then analyzed with an all-in-one fluorescence microscope “BZ-X800” (manufactured by Keyence Corporation). “Paraplast” is a registered trademark of Leica Biosystems Richmond.

### (Results)

As shown in the skin tissue section image after HE staining in FIG. 1, the epidermal layer of the three-dimensional skin model could be confirmed to be composed of the stratum corneum, the stratum granulosum (flattened cells including intracellular granules), the stratum spinosum (having spines around the cells), and the stratum basale (cubic form) in the same manner as the human skin tissue. The three-dimensional skin model also could be confirmed to have fibroblasts as a dermal layer arranged in a layered manner just below the stratum basale, showing basic morphological characteristics as a skin model.

### Histochemical Analysis 2 of Three-Dimensional Skin Model

### (Analysis Method)

A skin tissue of the three-dimensional skin model, which had been cultured in the vapor phase for 12 days by the above-described method, was further cultured for one week or more. The cultured skin tissue was stained with hematoxylin-eosin (HE) staining, and then analyzed with an all-in-one fluorescence microscope “BZ-X800”.

### (Results)

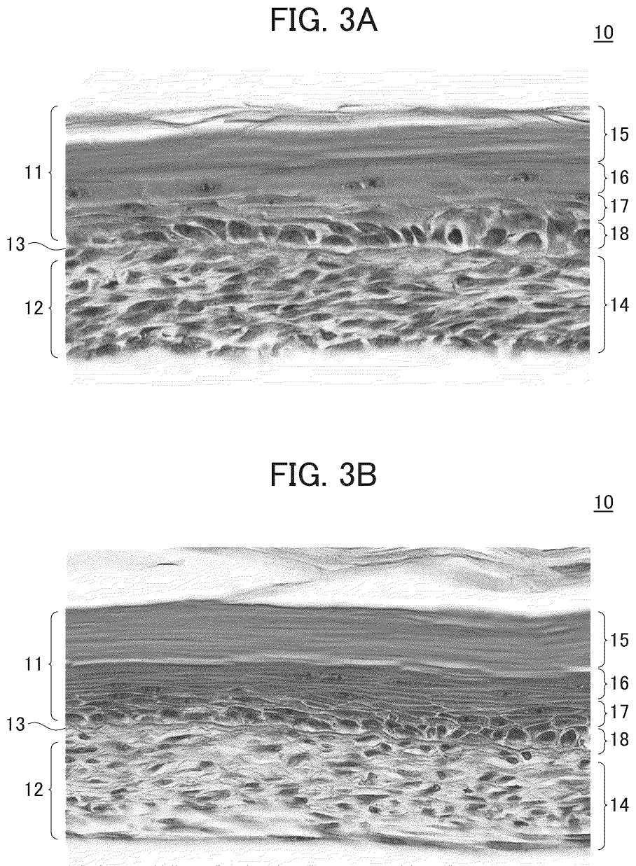

As shown in FIGS. 3A and 3B, comparing the skin tissue section image of the skin tissue (FIG. 3A) cultured in the vapor phase for 12 days and the skin tissue section image of the skin tissue (FIG. 3B) cultured for 20 days, the four-layer structure of the epidermal layer was maintained even after 20 days of culture. The three-dimensional skin model could be confirmed to be maintained for a longer period of time than in prior art.

### Histochemical Analysis 2 of Three-Dimensional Skin Model

### (Analysis Method)

A skin tissue of the three-dimensional skin model, which had been cultured in the vapor phase for 12 days by the above-described method, was fixed with Superfix KY-500 and embedded in Paraplast X-tra to prepare a paraffin-embedded section (skin tissue section). The prepared skin tissue sections were subjected to immunohistochemical staining using an antibody against a protein whose localization in the epidermal layer was revealed.

### (Results)

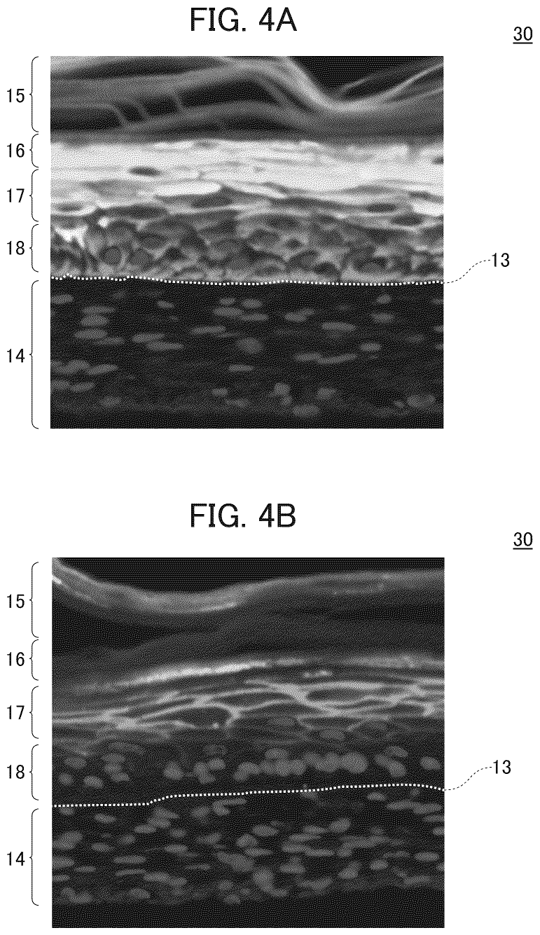

As shown in FIG. 4A, expression of cytokeratin 10 (CK10), which is a differentiation marker, was confirmed from the stratum corneum 15 to the stratum spinosum 17 of a three-dimensional skin model 30, and expression of cytokeratin 14 (CK14) was confirmed in the stratum basale 18. In addition, as shown in FIG. 4B, filaggrin (not shown), which is a component of keratohyalin granules, was confirmed to be expressed in the stratum granulosum 16 (granules in granular cells), and claudin 1 (not shown), which is an adhesion molecule constituting a tight junction, was confirmed to be expressed in the stratum spinosum 16 (periphery of spinous cells). In FIGS. 4A and 4B, the morphological characteristics of the three-dimensional skin model could be confirmed from the localization of the differentiation marker molecules.

### Barrier Function Analysis 1 of Three-Dimensional Cultured Skin Model

### (Analysis Method)

A sodium dodecyl sulfate (SDS), as a surfactant, solution prepared at a concentration of 0 to 0.5% (in increments of 0.1%) was applied to the epidermal surface of the three-dimensional skin model, which had been cultured in a vapor phase for 12 days by the above-described method. The resulting three-dimensional skin model was cultured in a CO₂ incubator at 37 °C for 1 hour. After culture, the SDS solution remaining on the epidermal surface was removed, the remaining SDS solution was washed away with phosphate buffered saline (PBS) (−), and recovery culture was further carried out in a CO₂ incubator at 37 °C for 24 hours. Cytotoxicity after culture was measured by Cytotoxicity LDH Assay Kit-WST (manufactured by Dojindo) using activity of a cytoplasmic oxidoreductase (LDH), which had leaked into the medium, as an index.

### (Results)

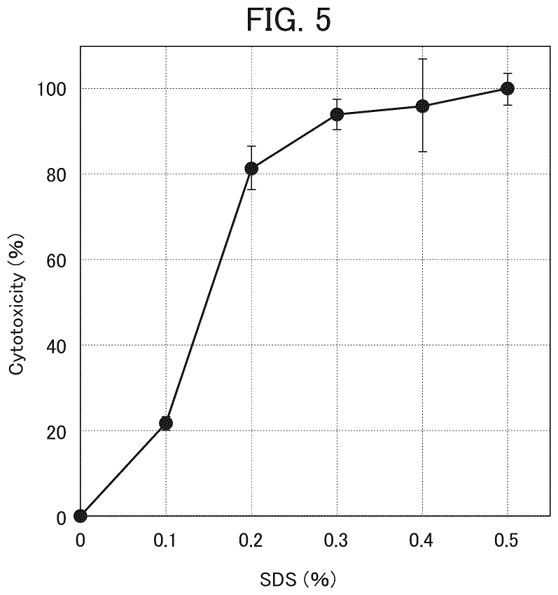

As shown in FIG. 5, since the cytotoxicity of the three-dimensional skin model showed an increasing tendency as the concentration of the SDS solution increased, the three-dimensional skin model was confirmed to have a barrier function in the epidermis.

### Barrier Function Analysis 2 of Three-Dimensional Cultured Skin Model

### (Analysis Method)

The transepithelial electrical resistance (TEER) of the three-dimensional skin model was measured over time, with the day when normal human dermal fibroblasts and normal human epidermal keratinocytes were seeded in inserts by the above-described method and vapor phase culture was started, as day 0.

### (Results)

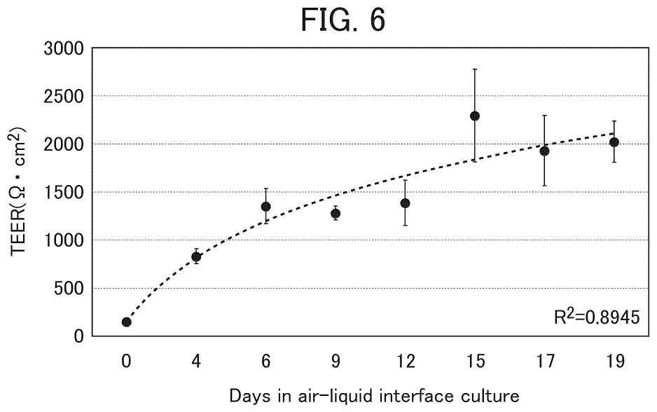

As shown in FIG. 6, the barrier function was confirmed to be enhanced along with the growth of the three-dimensional skin model, and to be maintained even after maturation of the three-dimensional skin model.

### Responsiveness Analysis 1 to Physiological Stimulus of Three-Dimensional Skin Model

### (Analysis Method)

A 10 μM solution of retinoic acid (ATRA) was applied to the epidermis (surface) of the three-dimensional skin model cultured in vapor phase for 12 days by the above-described method, followed by culture at 37 °C for 1 hour. After culture, the retinoic acid solution was removed, culture was further performed for 5 hours, RNA was extracted from the skin tissue, and gene expression analysis was performed using a real-time PCR method.

### (Results)

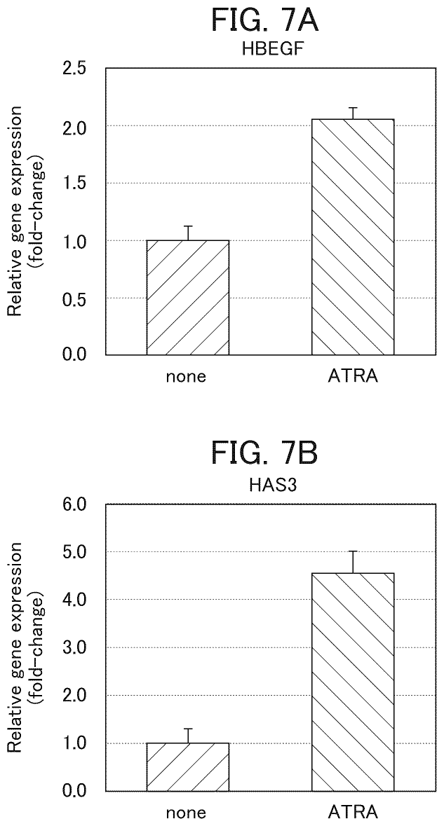

As shown in FIGS. 7A and 7B, the application of the ATRA solution was confirmed to induce expression of heparin-binding EGF-like growth factor gene (HBEGF) and epidermal hyaluronic acid synthase gene 3 (HAS3).

### Responsiveness Analysis 2 of Three-Dimensional Skin Model to Physiological Stimulus

### (Analysis Method)

A transforming growth factor β (TGFβ) prepared so as to have a final concentration of 10 nM was added to a medium of the three-dimensional skin model cultured in vapor phase for 12 days by the method described above, culture was performed for 48 hours, and gene expression analysis of the reconstructed three-dimensional skin model was performed. In addition, immunohistochemistry staining was performed using an antibody against a protein whose localization in the dermal layer was revealed.

### (Results)

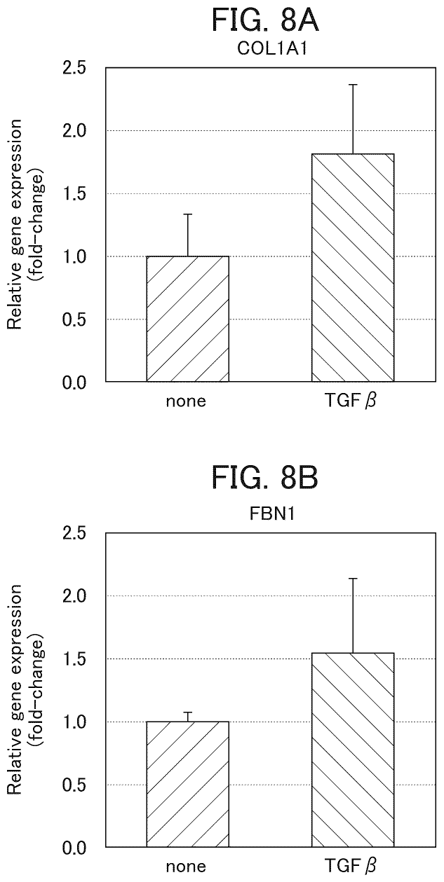

As shown in FIGS. 8A and 8B, induction of expression of genes of collagen fibers and/or elastic fibers of dermis, such as COL1A1 (type I collagen gene), FBN1 (fibrillin-1 gene), etc., could be confirmed.

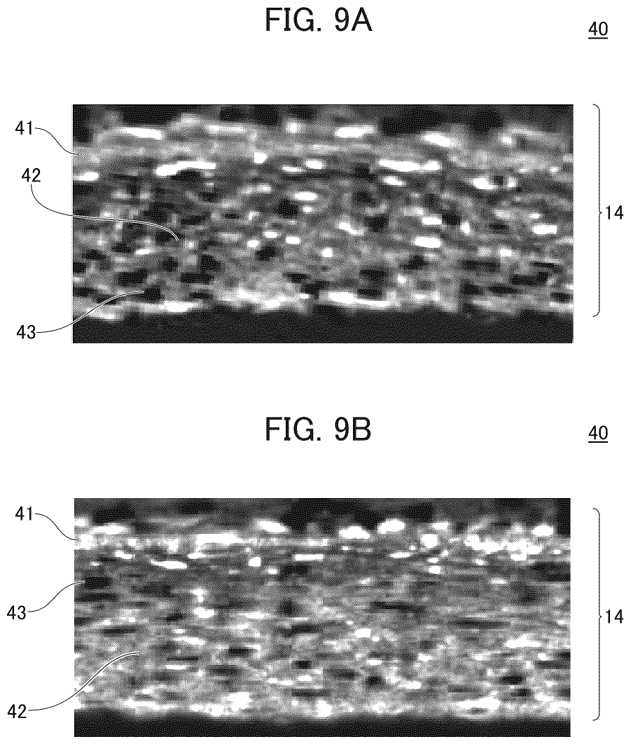

As shown in FIGS. 9A and 9B, when confirmation of expression of type I collagen (41) and fibrillin-1 (42) in the dermal fibroblast layer 14 (dermal layer) was carried out by immunohistochemistry staining, in the three-dimensional skin model (40) cultured by adding TGFβ, an increase in expression and a change in localization of fibrillar protein could be confirmed. This also confirmed that the reconstructed skin model was responsive to physiological stimuli.

### Response Analysis of Three-Dimensional Skin Models to Cosmetics

### (Analysis Method)

Commercially available cosmetics (skin care products) 1 to 5 were each applied to the epidermal surface of a three-dimensional skin model cultured in the vapor phase for 12 days by the above-described method, and culture was performed in a CO₂ incubator at 37 °C for 1 hour. After culture, the cosmetics were removed, and the three-dimensional skin models were further cultured for 5 hours. Then, RNA was extracted from the skin tissues, and the expression of epidermal genes (Filaggrin (FLG)/caspase 14 (CASP14)/ transglutaminase 1 (TGM1)/ocrudin (OCLN)/heparin-binding EGF-like growth factor (HBEGF)/lysosome protease cathepsin V (CTSV)/syntaxin (STX3)) and a dermal gene (hyaluronic acid synthase 2 (HAS2)) were analyzed by a real-time PCR method.

### (Results)

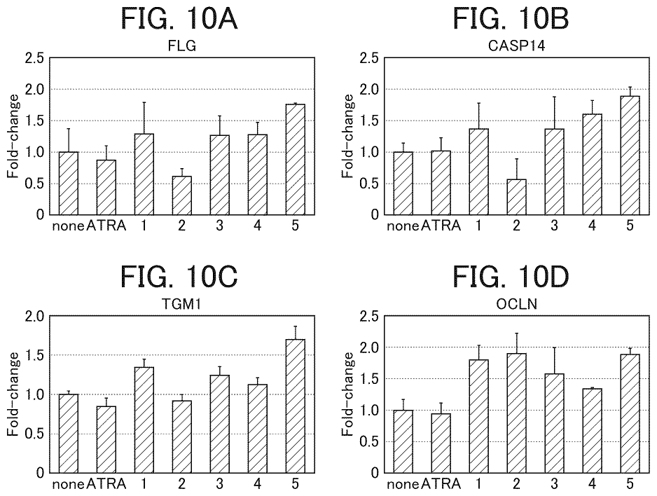

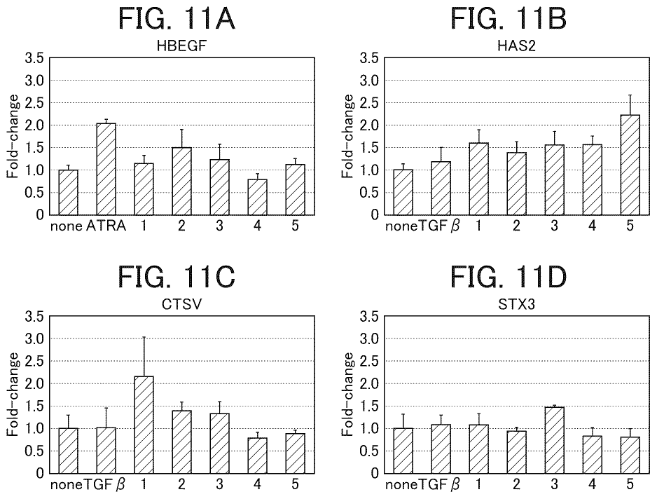

As shown in FIGS. 10A to 10D and FIGS. 11A to 11D, cosmetics 1 to 5 were confirmed to exhibit different responsiveness depending on the type of epidermal genes. As a result, a function of a product could be confirmed to be able to be evaluated by analyzing a three-dimensional skin model to which the product (cosmetic product or the like) is applied.

### Protein Expression Analysis of Three-Dimensional Skin Models

### (Analysis Method)

From the three-dimensional skin model cultured in the vapor phase for 14 days by the above-described method, only the epidermal layer was extracted to prepare an extracted protein solution, by using a general cell lysis solution. Protein expression analysis of a skin tissue was performed by western blotting using the prepared extracted protein solution.

### (Results)

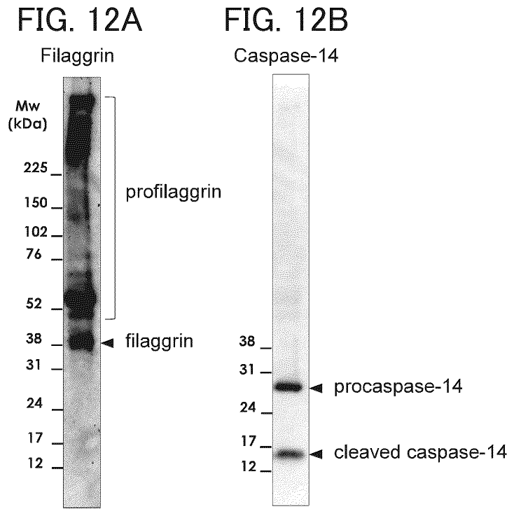

As shown in FIGS. 12A and 12B, western blotting confirmed the synthesis and metabolism of filaggrin and caspase 14 from the stratum granulosum.

### Responsiveness Analysis of Three-Dimensional Cultured Skin Model to Cosmetics in the Same Dosage Form

### (Analysis Method)

In order to evaluate the function in the same category (dosage form) of skin care products, serums (S001 to S016), lotions (L001 to L012), emulsions (E001 to E011) or creams (C001 to C012) of commercially available cosmetic products (skin care products) were applied to the surface of epidermis of the three-dimensional skin models cultured in a vapor phase for 12 days, and each skin model was cultured in a CO₂ incubator at 37 °C for 1 hour. After culture, the cosmetics on the epidermis surface were removed, and the three-dimensional skin models were further cultured for 23 hours or 47 hours. Then, RNA was extracted from the skin tissue, and expression of various epidermal genes (glucosylceramidase (GBA)/serine palmitoyltransferase long chain base subunit 1 (SPTLC1)/very-long-chain-fatty acid elongase 4 (ELOVL4)/transglutaminase 1 (TGM1)/occludin (OCLN)/heparin-binding EGF-like growth factor (HBEGF)/lysosomal protease cathepsin V (CTSV)/syntaxin 3 (STX3) and the like) and dermal genes (type I collagen a chain 1 (COL1A1)/fibrillin (FBN)/fibrin 5 (FBLN5)/latent TGFβ binding protein 4 (LTBP4)/hyaluronic acid synthase 2 (HAS2) and the like) was analyzed using a real-time PCR method.

### (Results)

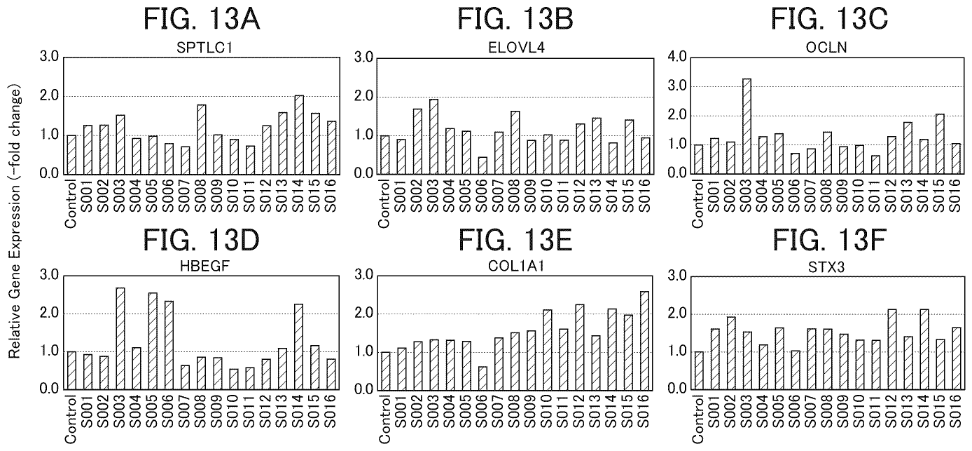

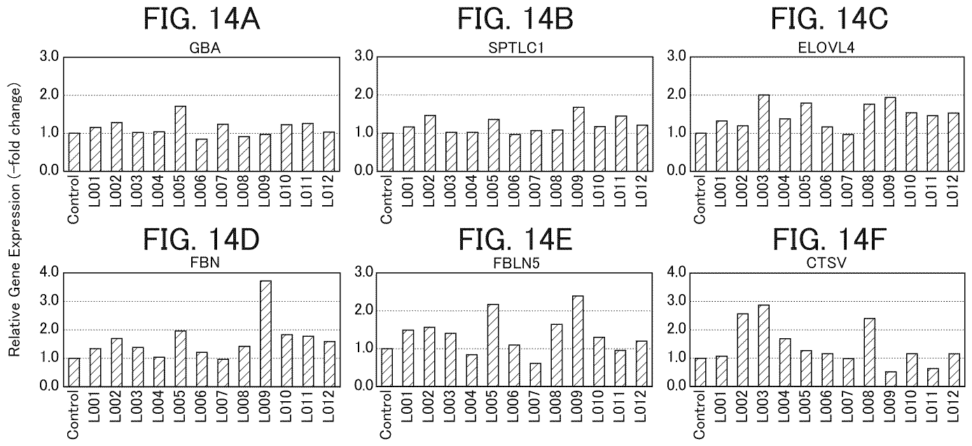

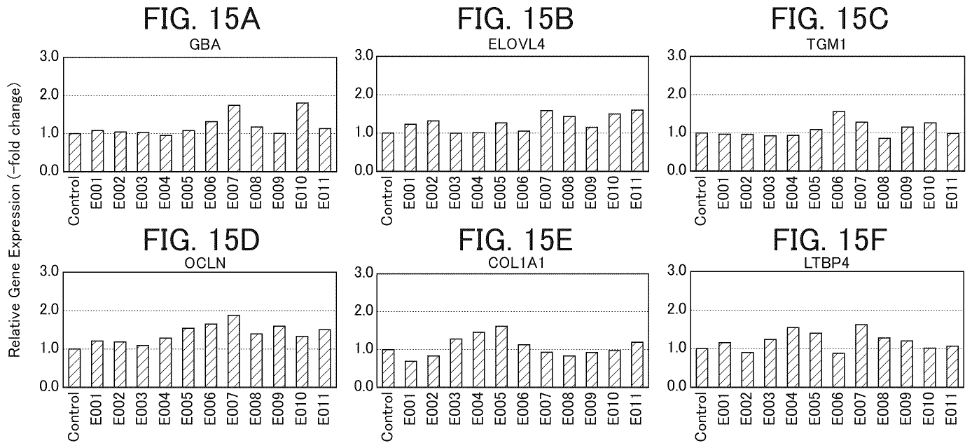

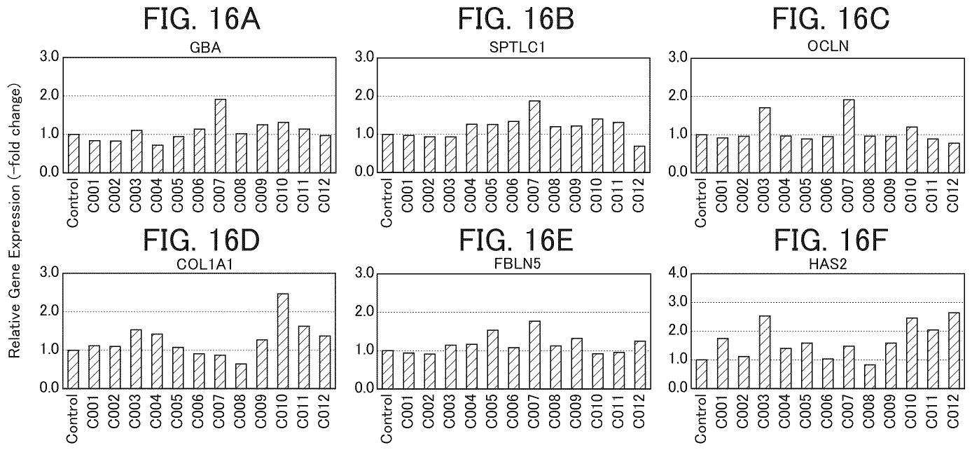

As shown in FIGS. 13A to 13F to FIGS. 16A to 16F, the epidermal genes and the dermal genes were confirmed to exhibit different responsiveness to the products of the serums (S001 to S016), the lotions (L001 to L012), the emulsions (E001 to E011), and the creams (C001 to C012), depending on the types of the products. As a result, a function of a skin care product could be confirmed to be able to be evaluated by analyzing a three-dimensional skin model to which the product (cosmetic product or the like) is applied.

### INDUSTRIAL APPLICABILITY

According to the present invention, shrinkage of the dermal layer can be suppressed and the structure of the epidermis and dermis can be maintained for a long period of time, and therefore, the present invention is effective for efficient sample evaluation of cosmetics and the like, the sample evaluation being performed using a culture container having many wells.

### EXPLANATION OF REFERENCE NUMERALS

10, 30, and 40 Three-dimensional skin models
11 First Layer, epidermal layer
12 Second Layer, dermal layer
13 Intervening membrane
14 Dermal fibroblast layer
15 Stratum corneum
16 Stratum granulosum
17 Stratum spinosum
18 Stratum basale
20 Culture container
21 Well
22 Support
23 Culture fluid
41 Type 1 collagen
42 Fibrin-1
43 Nucleus

1. A three-dimensional skin model comprising:
a first layer comprising keratinocytes or cells differentiated from keratinocytes;
a second layer comprising dermal fibroblasts; and
an intervening membrane interposed between the first layer and the second layer,
wherein the intervening membrane is a porous membrane comprising at least an extracellular matrix on a surface thereof.

2. The three-dimensional skin model according to claim 1, wherein the three-dimensional skin model comprises a collagen hydrogel in an amount of 0 w/v % or more and less than 0.6 w/v %, and v refers to a volume of the second layer.

3. A three-dimensional skin model, comprising:
an epidermal layer;
a dermal layer; and
an intervening membrane interposed between the epidermal layer and the dermal layer,
wherein the epidermal layer has a stratum corneum, a stratum granulosum, a stratum spinosum, and a stratum basale, and
the dermal layer has a multilayer structure and has a cell density of 2,000 cells/cm² or more.

4. A three-dimensional skin model, comprising:
an epidermal layer;
a dermal layer; and
an intervening membrane interposed between the epidermal layer and the dermal layer,
wherein the epidermal layer has a stratum corneum, a stratum granulosum, a stratum spinosum, and a stratum basale,
the dermal layer has a multilayer structure and comprises a collagen hydrogel in an amount of 0 w/v % or more and less than 0.6 w/v %, and
v refers to a volume of the dermal layer.

5. A culture container comprising: the three-dimensional skin model according to claim 1;
a plurality of wells comprising a culture fluid; and
a support portion that supports the three-dimensional skin model in each well and allows the culture fluid to communicate with the three-dimensional skin model on a second layer side or dermal layer side thereof.

6. The culture container according to claim 5, wherein the culture container has 12, 24, 48, or 96 wells.

7. A method of evaluating a sample, comprising:
culturing a three-dimensional skin model using the culture container according to claim 5;
administering a sample to the three-dimensional skin model during culture;
and evaluating a function given to the three-dimensional skin model by the sample by analyzing the three-dimensional skin model or a culture supernatant after a predetermined culture period elapses from the administration of the sample.

8. The method of evaluating according to claim 7, wherein the culture period is 6 days or more.

9. The method of evaluating according to claim 7, wherein the evaluating comprises, during the culture period, processing the three-dimensional skin model, administering same samples or different samples two or more times, or removing the administered samples.

10. The method of evaluating according to claim 7, wherein the three-dimensional skin model to which at least a sample is not administered has an epidermal layer having a stratum corneum, a stratum granulosum, a stratum spinosum, and a stratum basale, and has a dermal layer having a multilayer structure, after the culture period elapses.

11. The method of evaluating according to claim 7, wherein a plane area of an epidermal layer and a plane area of a dermal layer of the three-dimensional skin model after the culture period elapses satisfy a relationship of the plane area of the epidermal layer≤the plane area of the dermal layer, or the plane area of the dermal layer after the culture period elapses is 0.7 times or more the plane area of the dermal layer before the culture period elapses.

12. The method of evaluating according to claim 7, wherein the analysis comprises gene expression, protein expression, histochemical analysis, or skin barrier function analysis.
